(19) United States
(12) Patent Application Publication
(10) Pub. No.: US 2014/0343555 A1
(43) Pub. Date: Nov. 20, 2014

(54) MINIMALLY INVASIVE OSTEOTOMY DEVICE WITH PROTECTION AND CUTTING GUIDE

(71) Applicant: Martin Russi, Montevideo (UY)

(72) Inventors: Martin Russi, Montevideo (UY); Alejandro Texeira, Montevideo (UY)

(21) Appl. No.: 14/277,392

(22) Filed: May 14, 2014

(30) Foreign Application Priority Data
May 15, 2013 (UY) 34.810

(51) Int. Cl. A61B 17/15; A61B 17/00; A61B 17/14

(52) U.S. Cl. CPC A61B 17/155; A61B 17/141; A61B 17/00234
USPC 606/82

(57) ABSTRACT

The present invention is related to techniques for designing and constructing devices employed in surgical interventions, through minimally invasive techniques with protection and cutting guide generally comprising a pair of assembly components formed by a first main body and a second main body joined together at their mid-section (intersection area) and at the distal ends of the handles forming a cross tong-type system.

### FIELD OF THE INVENTION

The present invention relates to techniques for designing and constructing devices used in surgical interventions, particularly osteotomies, by minimally invasive techniques with protection and cutting guide.

### BACKGROUND OF THE INVENTION

Nowadays, when a person is in need of a surgical intervention which procedure requires cutting a bone structure, the surgeon generally employs a wide access to enable a good visualization of the surgical field, as well as to protect the adjoining structures.

To do this, the surgeon has to make significant incisions in the skin, muscles and other tissues of the patient, placing separators and protective material such as compresses or gauze wicks in order to protect noble structures such as vessels and nerves which may otherwise be injured.

There are various devices in the state of the art to make cuts in bone structures, such as the “Universal cutting guide for total knee arthroplasty intervention” of patent application EP 2092899, in which a minimally invasive cutting guide for use in tibial and femoral resections is described. The guide has a curvature which is similar to the generic proximal tibia and distal femur. This device has a difficult, time-consuming mounting and it does not provide safe cutting elements.

Another invention which attempts to solve the problem of making safe and accurate cuts is that of US patent US 2005267484 which shows a surgical guide to orient and stabilize a saw during a trochanteric osteotomy. The guide includes a linear slot and two slots at oblique angles opposite the linear slot, which are adapted to cooperate with a surgical saw. This guide may include a plurality of anchoring sites for fixing and securing the guide to the femur while making the cut. This device also has the deficiency of difficult mounting and of not providing enough cutting elements, since although it provides a guide for the saw path, there is no stop or end point for the cut and thus there is the risk of damaging the structures if the saw goes beyond the bone.

### BRIEF DESCRIPTION OF THE FIGURES

The novel aspects characteristic of the present invention will be particularly established in the appended claims.

However, the invention itself, given its structure and operation, in conjunction with other objects and advantages thereof, will be better understood in the following detailed description of a preferred embodiment when read in connection to the accompanying drawings, wherein:

FIG. 1 is a front perspective view of a preferred embodiment of the osteotomy device of the present invention.

FIG. 2 is a bottom perspective view of the first main body which is part of the surgical intervention device shown in FIG. 1.

FIG. 3 is a bottom perspective view of the second main body which is part of the surgical intervention device shown in FIG. 1.

FIG. 4 is a front perspective view of the surgical intervention device shown in FIG. 1, positioned around the bone, with the cutting device in operation.

FIG. 5 is a front perspective view of the surgical intervention device shown in FIG. 1, interconnected to a spatial positioning device which secures it to the bone.

FIG. 6 is a front perspective view of a first cross tong-type variant of the minimally invasive osteotomy device, which is shown positioned around the bone.

FIG. 7 is a bottom perspective view of the minimally invasive osteotomy device shown in FIG. 6, where the two main bodies are shown when not coupled.

FIG. 8 is a front perspective view of a first variant of the surgical intervention device, positioned around the bone, with the cutting device in operation.

FIG. 9 is a bottom perspective view of the surgical intervention device shown in FIG. 8, where all of its components are appreciated when not coupled (first main body, second main body, third coupling element and cutting guide, fourth cutting guide element for bit cuts, cutting elements).

FIG. 10 is a side view of a third variant of the minimally invasive osteotomy device, consisting of only one main body where the telescopic unfolding of the inner sheet of the protective element is visualized.

FIG. 11 is a side view of the minimally invasive osteotomy device shown in FIG. 10, wherein the telescopic sheet of the protective element has not been unfolded.

FIG. 12 is a side view of the distal end of the minimally invasive osteotomy device shown in FIG. 10, where an L-shaped, quick coupling of the telescopic system is evidenced.

FIG. 13 is a front perspective view of the minimally invasive osteotomy device shown in FIG. 10, which is positioned around the bone, with the cutting guide element coupled.

FIG. 14 is a front perspective view of an additional variant of the minimally invasive osteotomy device in which the two Hohmann-type main bodies are connected directly to the cutting element.

FIG. 15 is a profile view of the minimally invasive osteotomy device shown in FIG. 14.

FIG. 16 is a profile view of a variant of the osteotomy device formed by only one element, which is incorporated to the cutting element.

FIG. 17 is a front perspective view of the minimally invasive osteotomy device shown in FIG. 16.

FIG. 18 is a profile view of another embodiment of the minimally invasive tong-type device, wherein the cut is made laterally by a swing sheet saw incorporated to the device.

FIG. 19 is a front view of another additional embodiment of the minimally invasive osteotomy device formed by a tong-type system consisting of only one element, which via a threaded gripping system enables fixation to the bone, demarcating the cut by a lateral stop and/or a stop anterior to the bone plane.

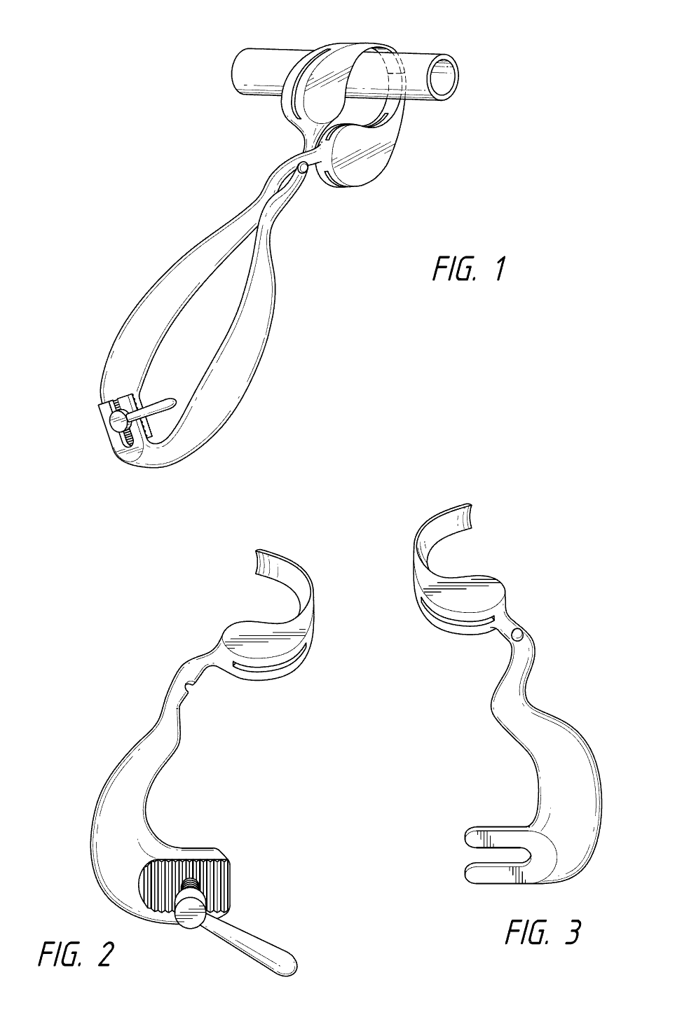

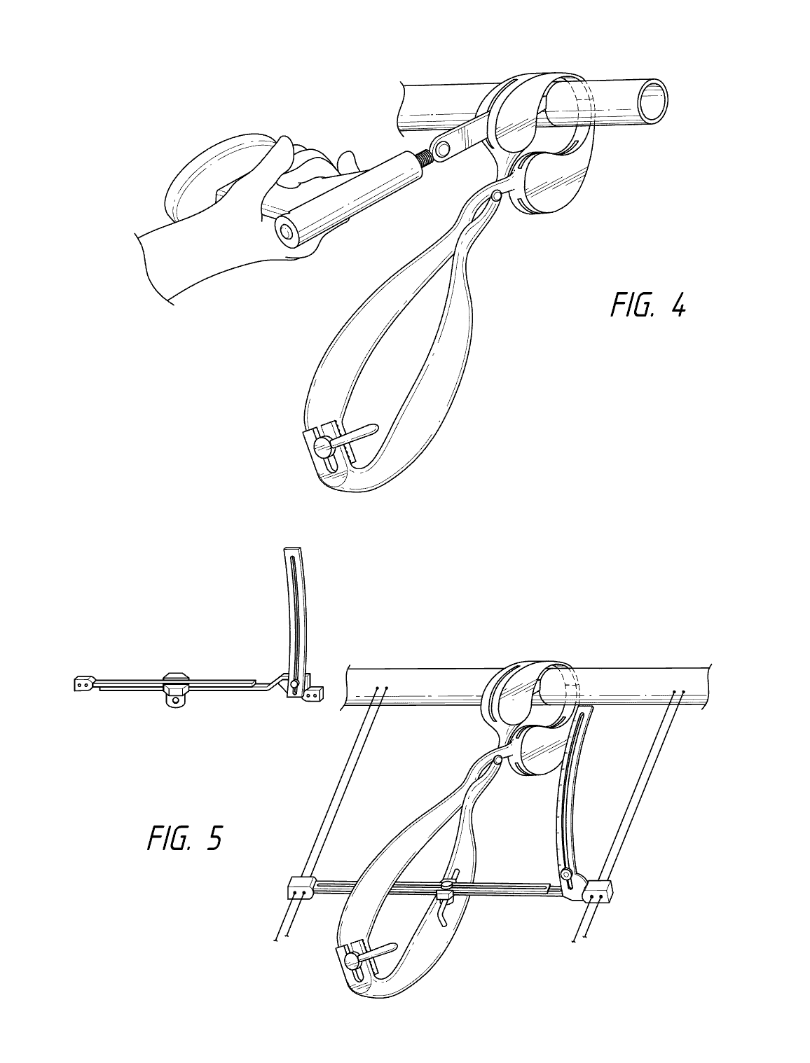

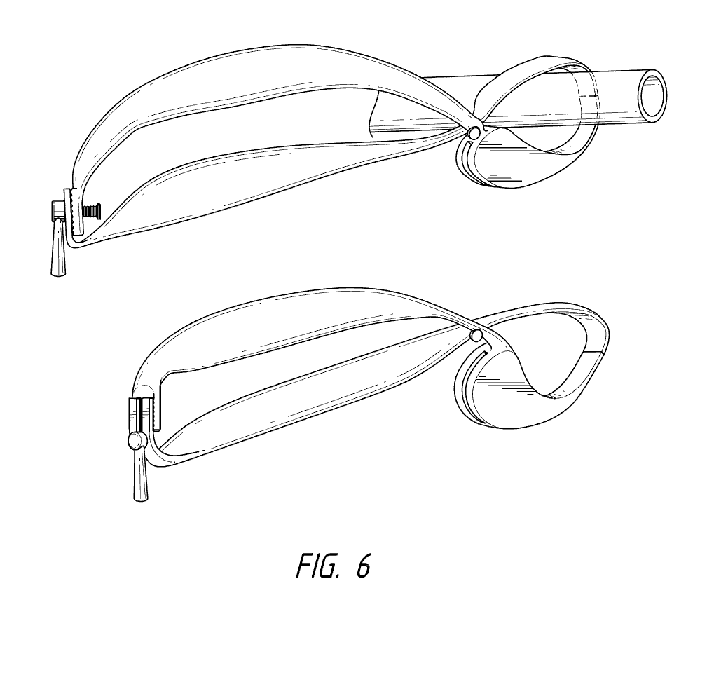

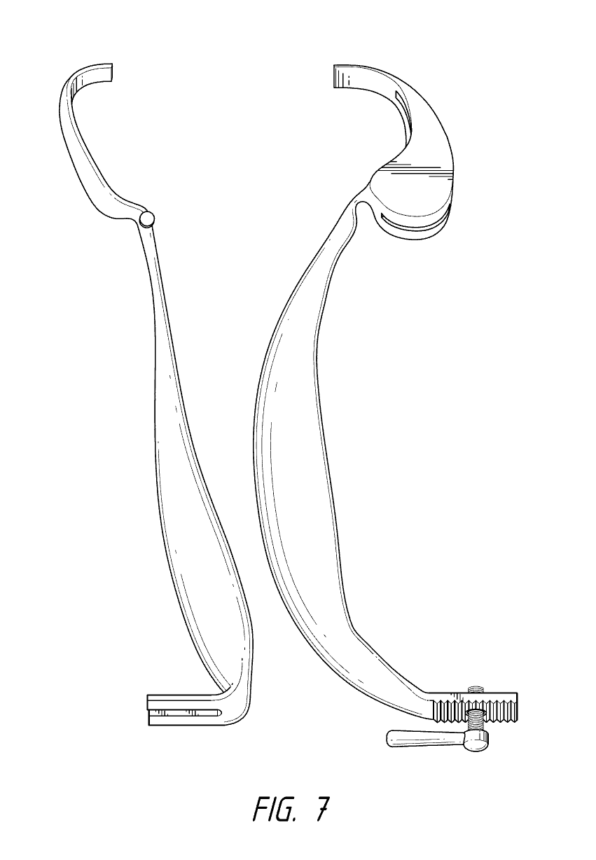

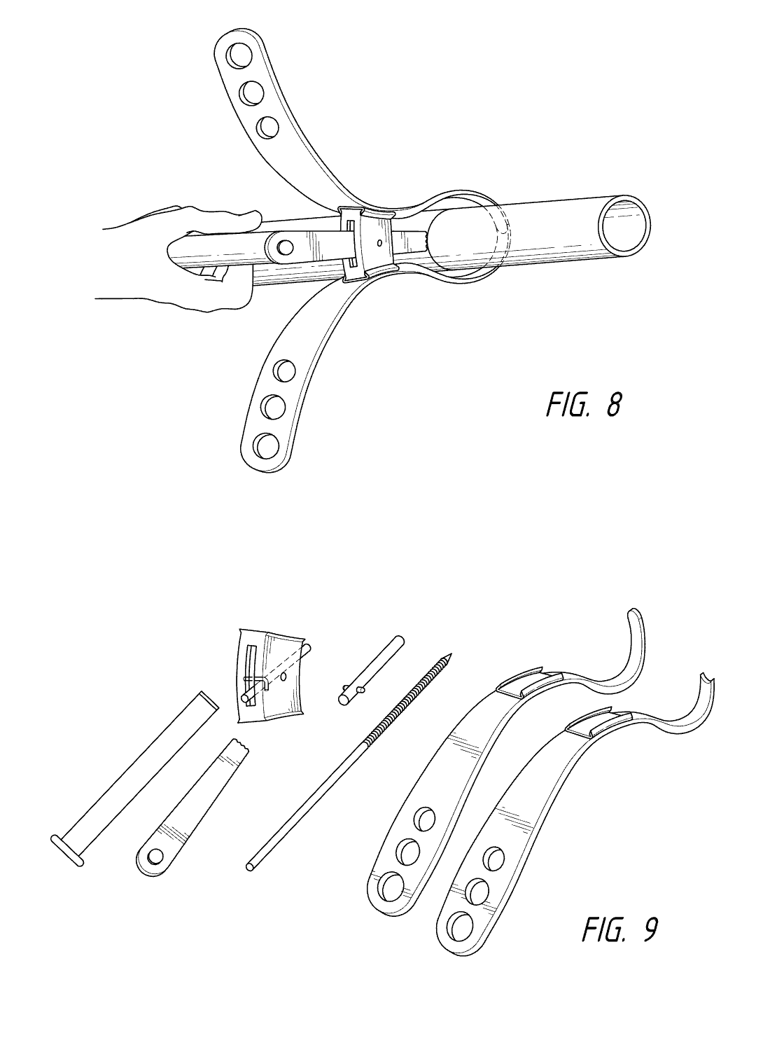

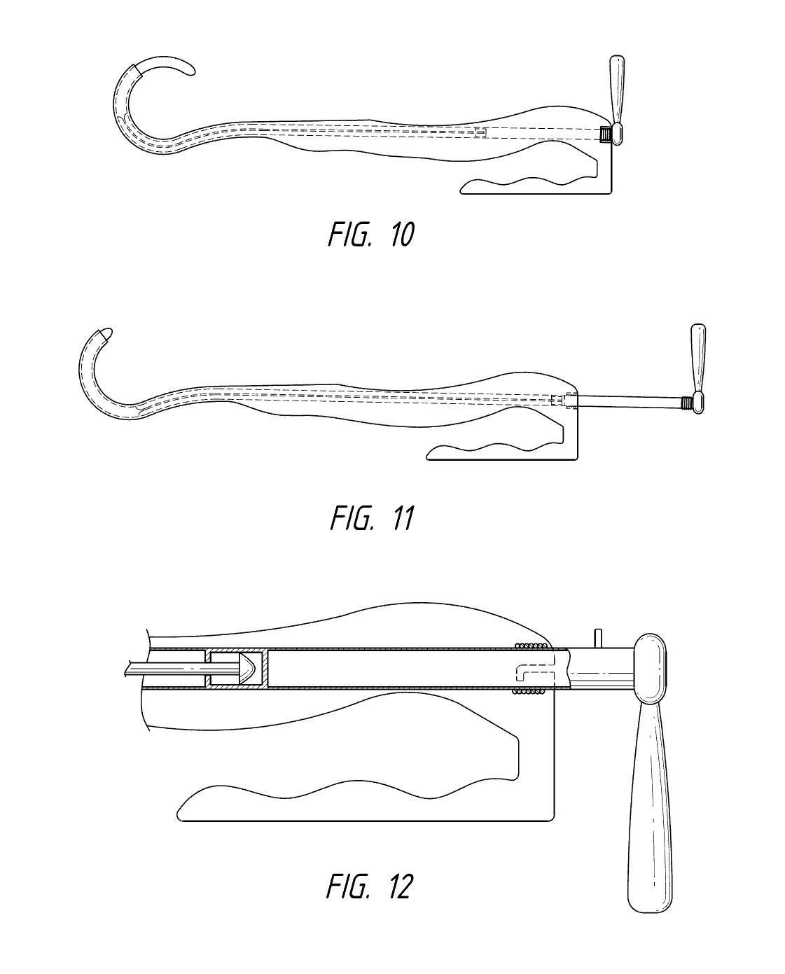

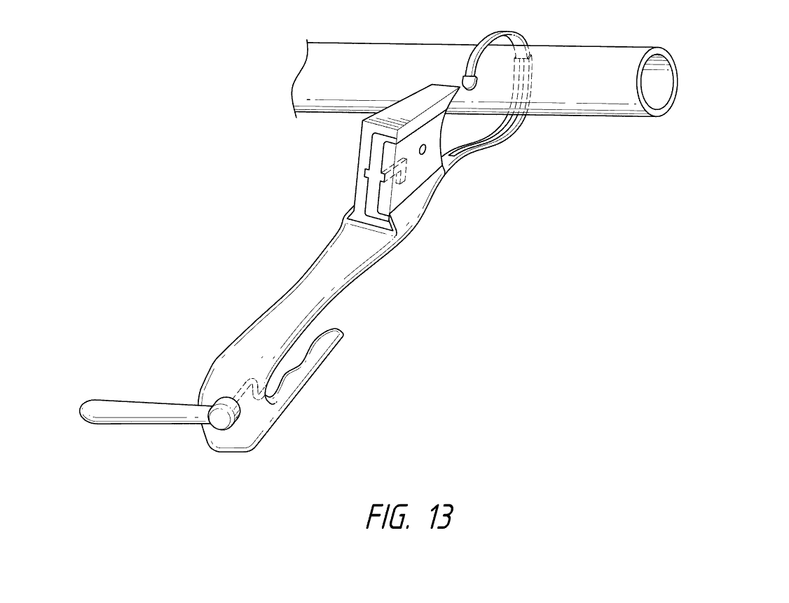

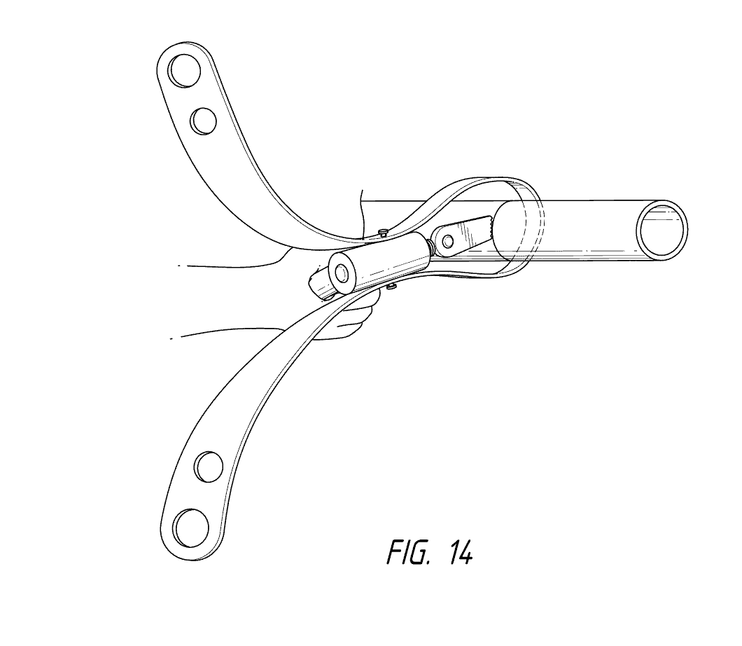

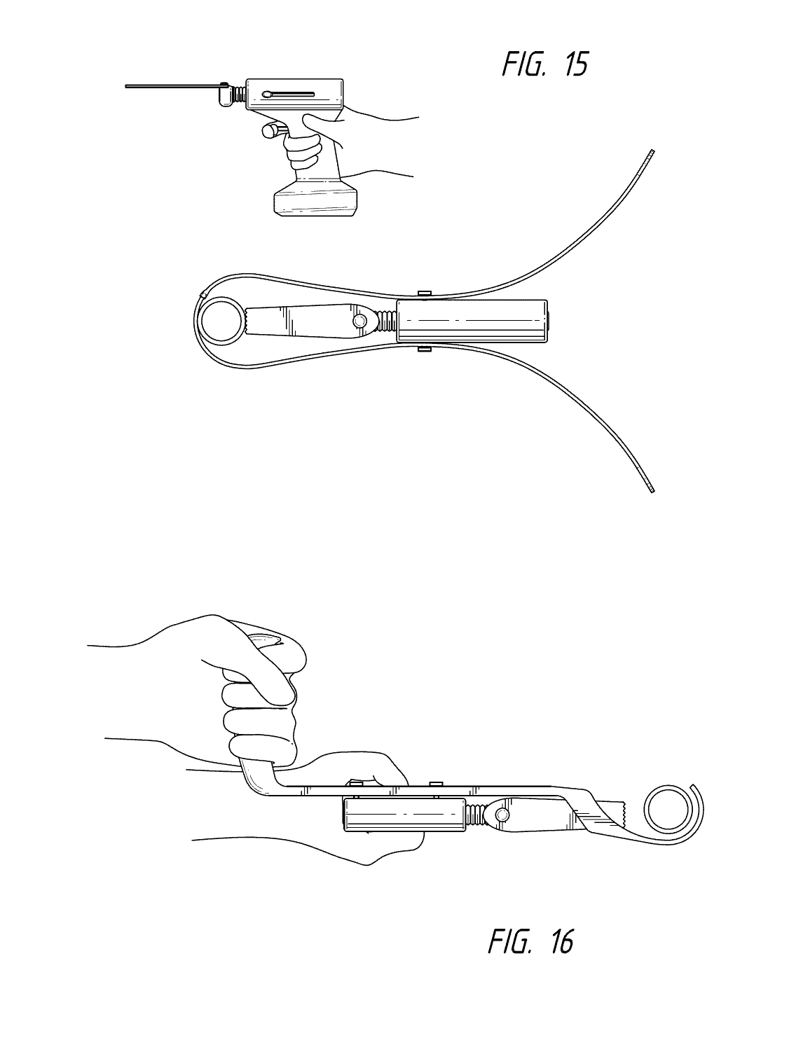

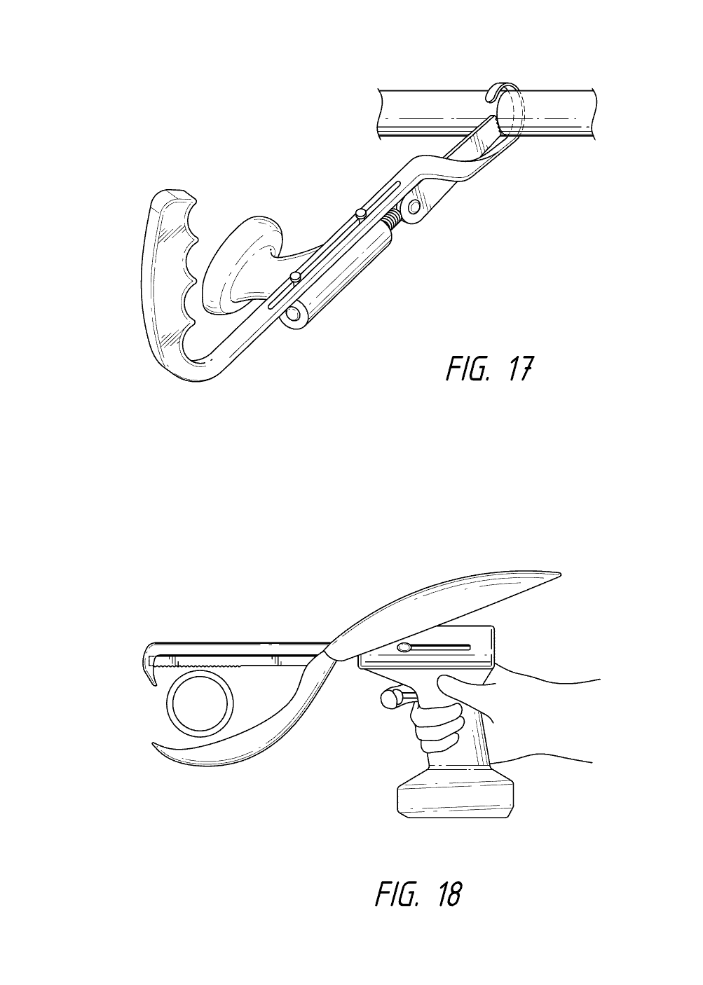

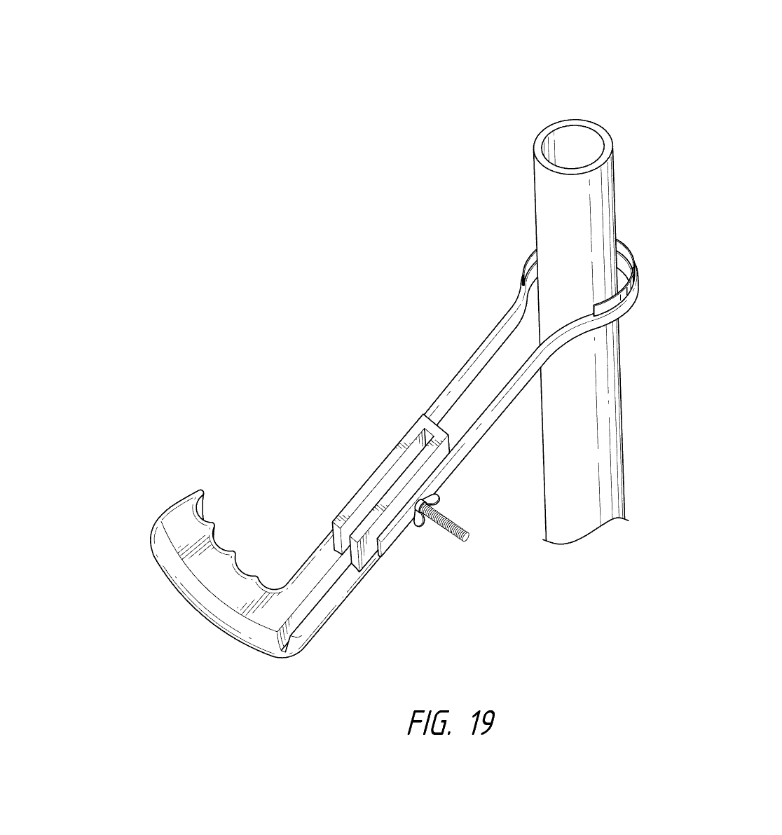

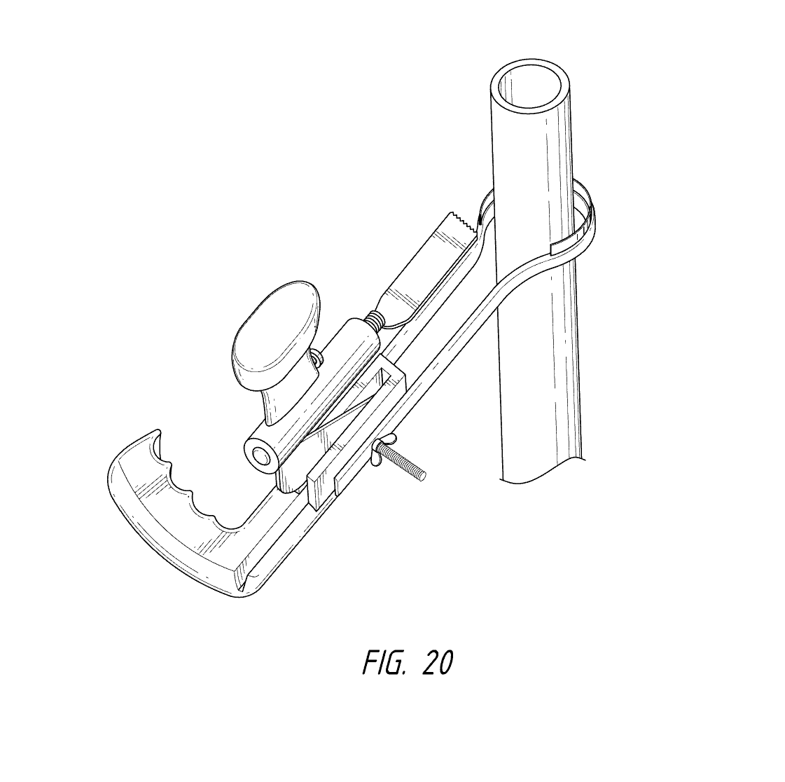

The cutting element is coupled thereto as shown in FIG. 20.

### BRIEF DESCRIPTION OF THE INVENTION

In order to suppress the disadvantages of the prior art, a minimally invasive device for surgical interventions has been developed, generally comprising a pair of assembly components formed by a first main body and a second main body joined together at their mid-section (intersection area) and at the distal ends of the handles forming a cross tong-type system.

A cutting element, which in this model is the sheet saw.

The first main body consists of a grip handle followed by a first protective element.

An anchoring area is located at the most distal part of the first grip handle, which presents a threaded bore where the gripping element is threadedly inserted, which element is fixed in this handle.

At the most proximal part of the first handle there is an intersection area with the second handle, where there is an anchoring area in the form of a hitch. Further, the first protective element presents, at its most distal, solid portion, a central, vertical slot following the axis of the handle which, together with a slot located at the second protective element, is used as a cutting guide for the sheet saw. At its most proximal section, the first protective element has a semicircular shape from a side view, which embraces the inner section of the bone. Said section is formed by a semi-tubular shaped sheet from a cross-sectional view.

This most proximal section of the protective element generates a stop for the bone cut, demarcating a safe cutting area.

The gripping element consists of a central handle with a plurality of channels on its outer surface which increase the grip of such central handle. At its lower end there is a head, from the inner side of which a threaded protuberance extends having a smaller diameter than said head, which is inserted into the bore of the anchoring area of the first main body. This gripping element remains fixed to the first handle by virtue of a rivet at the other side of the bore, which enables the tightening or loosening of the threaded protuberance without it unwinding completely.

The second main body consists of a second grip handle followed by a second protective element.

At the most distal portion of the second grip handle there is a U-shaped coupling area where the coupling element present at the most distal portion of the first handle is inserted.

At the most proximal portion of the second handle, the intersection area with the first handle, there is a coupling area in the form of a hitch.

The second handle is followed by the second protective element which has similar features to the first protective element.

At its most distal section, the second protective element has a solid morphology with a vertical, central slot guiding the bone cut, at its most proximal section it embraces the bone with its inner side as a semicircular plate from a profile view, having a semi-tubular shape from a cross-sectional view, which section generates a stop for the bone cut.

A variant of this osteotomy device in the form of a cross tong has the same features as described above with the difference that the cross coupling is offset the access, making it possible for only one main body to present the solid cutting guide on its protective element, leaving the protective element of the other main body shaped as a curved sheet from a profile view and as a semi-tube from a cross-sectional view, functioning as a bone cut stop or limit without requiring a solid guide structure.

A second variant of this osteotomy device consists of a pair of assembly elements formed by a first main body and a second main body joined together at its proximal section and joined by a third element at the mid-section.

A cutting guide element which may be part of the third element or which may be inserted in it, and a cutting element which may be an oscillating sheet saw, bit or chisel.

The first main body is formed by a first grip handle followed by a first protective element.

The anchoring area for the third element is located on the inner side of the first handle. Further, the first protective element consists of a wide plate shaped as a curved hook from a profile view and as a semi-tube from a cross-sectional view, having at its proximal end a solid blunt point in the shape of an elevator, facilitating its passage around the bone, said point also determining a coupling or fitting area with the proximal end of the protective element of the second main body.

The second main body is formed by a second grip handle and a second protective element.

At its inner side, the second grip handle presents an anchoring area for the third element.

The second protective element presents features which are similar to the first protective element with the variant that, from a profile view, instead of having a hook shape it has a circular shape, a fourth of the circumference ending in a cupped coupling area to receive the proximal end of the first protective element.

When the two protective elements positioned around the bone are coupled, they generate a stop or action limit for the cutting element.

This pair of assembly elements are joined at the inner area of their handles by a third element in the form of a solid cube with a central hollow cavity in the shape of a vertical, adjustable slot which enables guiding of the cut.

The vertical slot is adjustable since one of its walls is mobile by means of a threaded system for a hex key.

It is important to adjust the slot and to prove it with the cutting device to be used prior to placing it in the osteotomy device.

At the mid-section of this vertical slot there are two lateral grooves shaped like a horizontal L with its long arm in the direction of the cut and its short arm in a downwardly oriented vertical position. These L-shaped grooves enable the fitting of a fourth element.

The tube-shaped fourth element, which acts as a guide in the case of a bit osteotomy, presents two axes, one on each side, which are inserted into the horizontal L-shaped grooves of the third element.

Once in place, this fourth element can rotate on its lateral axes guiding the perforations of the bit in the bone.

The cutting element can be a sheet saw, bit or chisel.

In an additional embodiment of the present device, it consists of only one main element, having a grip handle followed by the protective element.

The protective element consists of two sheets, an inner one and an outer one, semicircular from a profile view and semi-tubular from a cross-sectional view.

The outer sheet has a concave oriented hitch area or flange, for the inner sheet to be able to slide without deviating from its lane.

The inner sheet presents a coupling fixed to a sliding element.

The sliding element, which may be a sling or strip, runs across the interior of the handle, having a mushroom-shaped rivet at its distal end, which is housed in a cylindrical cavity wherein the most proximal side has a central opening through which the sliding element passes, and the cap-shaped most distal side is followed by a solid cylinder having a length such that the sliding element is not exposed out of the handle when the inner sheet of the protection element is not unfolded. This solid cylinder is distally continued with a handle coupling section, then a head with a central grip handle.

The handle coupling section may be a threaded section (when the coupling to the handle is threaded) or a cylinder with a vertical arm (when it is an L-shaped, quick coupling).

Once this device is in place, the inner sheet of the protective element is unfolded like a telescope, generating a protection around the bone without the need of coupling two elements, which results in a simpler, less invasive procedure.

The unfolding sheet of the protective element (inner sheet) presents, at its proximal end, a blunt point in the form of an elevator protruding from the second sheet, which enables the threading of the device around the bone. In a variant in which this telescopic system generates a greater unfolding of this unfolding sheet, it presents a central slot in its most distal end (the portion embracing the outer section of the bone) which aligns with the cutting guide element enabling the passage of the cutting element through this section.

The inner side of the handle presents a hitch area for the solid cutting guide element having similar features to that described above.

This cutting guide element enables the adjustment of the slot for different sheet saws, as well as for guiding the cut made with bit and chisel.

In another version, the guide element may already be incorporated in the handle, without the need for coupling. Once the minimally invasive osteotomy device is positioned, it can be fixed to the bone via a fixation device with Kirschner nails which also enables the spatial positioning of the cutting device, being able to adjust the bone cut in the sagittal, coronal and rotational planes.

This cutting adjustment and fixation device consists of a horizontal, straight arm which follows the bone axis, which is formed by two sheets and presents a central sliding channel running through its entire length, one of these sheets having an anchoring area for a Kirschner nail at one of its ends; the other sheet has, at its opposite end, a curved arm having a longitudinal central channel presenting an anchoring area for a Kirschner nail at one of its ends; and an anchoring device employed to secure the two sheets superimposed one another and the element for the spatial positioning to the osteotomy device.

The ends of this device having anchoring sites may vary their morphology, making it possible to use one, two or several Kirschner nails.

In an additional embodiment, the minimally invasive osteotomy device is formed by two symmetrical, Hohmann-type main bodies, constituted by a handle which from a side view is curved and concave on its outer side, having an area of coupling to the third element on its inner side (convex).

The handles of both main bodies are followed proximally by the protective elements. These protective elements have a curved shape from a side view, with inner concavity, and a semi-tubular shape from a cross-sectional view.

Once both Hohmann-type main bodies are positioned around the bone, they are coupled to the third element which is the oscillating part of the sheet saw, which can make side rotational and cut deepening moves, generating a safe cut without the risk of going deeper than necessary or deviating.

With this model, there is no need for a cutting guide since the cutting device itself is part of the minimally invasive osteotomy device.

Another version of the minimally invasive osteotomy device is that in which the device is coupled to the oscillating cutting saw, consisting of only one element.

Said element consists of a handle followed by a cutting protection element in the form of a hook.

The handle is formed by a solid body having two coupling areas for the oscillating sheet saw, wherein said coupling enables the saw to slide over the handle thus deepening the cut to a certain set limit.

At the distal end of the handle there is an ergonomic area for a better grip of this device.

At its proximal end, the handle is followed by two vertical plates demarcating a vertical slot where the sheet saw is hitched or positioned prior to initiating the cut. These vertical sheets finish proximally as a wide plate setting the limit of the slot, which is shaped like a hook from a side view and like a semi-tube from a cross-sectional view.

This wide plate is the cutting protective element which, at its proximal end, finishes in a solid blunt point in the form of an elevator.

Another embodiment in which the protection element is incorporated to the cutting device consists of a tong-type system formed by two main elements.

The first main element, formed by a handle having the saw motor and the swinging cutting mechanism thereof (similar to a sternum swing saw) incorporated, followed distally by the sheet saw with protection on the back and proximally as a hook.

At its mid-section, this first element has a hitch for the second main element.

The second main element consists of a handle, which is proximally followed by a cutting protection area limiting the cutting action of the saw present in the other main element.

In an additional embodiment in which the osteotomy device presents a coupling for the cutting element, it consists of a tong-type system formed by only one element wherein such system has two side arms which are coupled to the same handle, wherein between the two side arms there is a closing or opening threaded system, which system stays fixed to one of the handles and has a threaded seal with a butterfly for the other handle.

The solid handle follows an axis perpendicular to the axis of the arms, having a suitable morphology to be grabbed with one hand.

The handle is proximally followed by two straight, side arms which proximally continue in a semicircular shape with inner concavity, the ends of which finish as a sharp, jagged surface for gripping the bone plane.

The semicircular sections of the handles have, on their back, a flange or wall which provides a stop for the sheet saw, demarcating the cut on the lateral section.

On the back, the distal portions of the handles have a plate with a central slot following the axis of the handles, wherein a keel present at the back of the sheet saw is inserted.

This cutting guide slot enables deepening of the saw up to a predetermined stop at the level of the ends of the tong arms which are firmly secured to the bone.

Such slot may be adjustable in order to enable a greater or lesser deepening of the cut depending on the diameter of the bone, as well as on the level of deepening of the bone gripping side arms.

It should be noted that the structure of the osteotomy device which is the subject of the present invention enables its use on long bones, especially femur or tibia; in procedures such as bone lengthening and axial corrections of the limbs, as well as flat or short bone osteotomies, if deemed adequate.

### OBJECTS OF THE INVENTION

Considering the deficiencies of the prior art, it is an object of the present invention to provide a minimally invasive device for surgical interventions, which may be used in osteotomy procedures in a practical and simple way.

An additional object of the present invention is to provide a minimally invasive device for surgical interventions which minimizes the time of surgery and internal damage to the patient.

It is another object of the present invention to provide an osteotomy device enabling interventions which are safer and less invasive for the patient, with positioning elements demarcating a predetermined safe cutting area.

Another object of the present invention is to provide a minimally invasive osteotomy device, the design and construction of which are ergonomic so as to facilitate handling thereof by the medical team.

Still another object of the present invention is to provide an osteotomy device which enables guiding the cut so that it is made in the exact place and in the exact way as planned.

A further object of the present invention is to provide a minimally invasive osteotomy device having means of fixation to a bone structure, which can vary in number and distance, and a spatial positioning element which enables the adjustment of the cut of said bone structure in the three planes: sagittal, frontal and rotational.

The object is to create a device which enables guiding of the bone cut while demarcating a safe cutting zone, without being able to deviate from these limits when using a sheet saw or bit and chisel. Thus, injuries caused by deviations of the cutting element or by deepening past the bone plane are prevented.

While preferred embodiments of the present invention have been described and shown in the foregoing description, it should be emphasized that numerous modifications thereof are possible without departing from the true scope of the invention, such as modifying the disposition of the grip handles and the coupling elements, modifying the profile of the different elements composing the present device, modifying the gripping element, etc. Thus, the present invention should not be limited except by the requirements of the prior art and the appended claims.

1-29. (canceled)

30. A minimally invasive osteotomy device comprising a cutting guide, a soft part protection, at least one main element that comprises a handle; a positioning section for embracing a bone, wherein the position section comprise an element for securing the bone which enables the adjustment of the cut in the rotational, frontal and sagittal planes and maintains the bone fragments after the cut; and a cutting element.

31. The minimally invasive osteotomy device according to claim 30, comprises at least one pair of assembly elements formed by a first main body and a second main body, joined together at their respective mid and distal sections; a gripping element which is inserted at the distal end of both the first main body and the second main body, wherein the gripping element assists in keeping the first main body and the second main body together; a cutting guide; and a soft part protective element in the first main body and the second main body; and a cutting element acting in the interior of the cutting guide.

32. The minimally invasive osteotomy device according to claim 31, wherein the coupling of the first main body with the second main body in their mid and distal section forms a one circuital body shaped like a horizontal 8, wherein the proximal portion thereof embraces the bone generating a limit or stop for the cutting element.

33. The minimally invasive osteotomy device according to claim 32, wherein the first main body comprises a first grip handle extending from the base of the distal end of the device to the mid coupling area of said first main body; and a first protective element extending proximally to the intermediate coupling area of the first main body to the proximal end thereof.

34. The minimally invasive osteotomy device according to claim 33, wherein the first grip handle is a solid body having a hitch area in the proximal portion thereof and having an anchoring area in the distal end thereof, which presents a jagged homogeneous surface in its outer side and a threaded bore located in its center, and housing a gripping element therein.

35. The minimally invasive osteotomy device according to claim 33, wherein the first protective element is formed, at its most distal portion, by a solid structure with a central, vertical slot which is employed as a cutting guide; and at its most proximal section, the first protective element has a semicircular shape that embraces the inner section of the bone, said section is formed by a semi-tubular shaped sheet and generates a stop for the bone cut.

36. The minimally invasive osteotomy device according to claim 32, wherein the second main body comprises a second grip handle extending from the base of the distal end of the device to the proximal coupling area with the first main body; and a second protective element followed by the second grip handle and extending proximally to the mid anchoring area to the proximal end of said second proximal body.

37. The minimally invasive osteotomy device according to claim 36, wherein the second grip handle is a solid body having a U-shaped coupling element in its most distal portion, the inner side of which has a jagged homogeneous surface corresponding in shape and size to the jagged homogeneous surface of the anchoring area of the first grip handle.

38. The minimally invasive osteotomy device according to claim 36, wherein the second protective element is formed, at its most distal portion, by a solid structure with a central, vertical slot which is employed as a cutting guide for a sheet saw; and at its most proximal section, the second protective element has a semicircular shape, which embraces the inner section of the bone, said section is formed by a semi-tubular shaped sheet and generates a stop for the bone cut, demarcating a safe cutting area.

39. The minimally invasive osteotomy device according to claim 31, wherein the gripping element comprises a central handle, preferably cylindrical, on the outer surface of which there is a plurality of channels inter-crossing one another to form a coarse surface which increases the grip of said central handle; and a head located at the end of the central handle and positioned perpendicular to it, presenting a preferably cylindrical contour, having a threaded cylinder extending from the inner side thereof with a smaller diameter than said head, which is inserted into a bore of the anchoring area of the first handle, and at the other side of the bore this threaded cylinder has a rivet to prevent this gripping element from unwinding from the handle.

40. The minimally invasive osteotomy device according to claim 30, comprises at least one pair of assembly elements formed by a first main body and a second main body joined together by their respective proximal ends; a gripping element which is inserted into an intermediate portion; a cutting guide element which is coupled to the gripping element; and a bone cutting element.

41. The minimally invasive osteotomy device according to claim 40, wherein the first main body is formed by a first grip handle followed by a first protective element; further comprising an anchoring area for a third element located on the inner side of the first handle; and wherein the first protective element comprises a wide plate having a curved hook shape and a semi-tubular shape having a coupling area at its proximal end.

42. The minimally invasive osteotomy device according to claim 40, wherein the second main body is formed by a second grip handle and a second protective element, wherein the second grip handle presents an anchoring area on its inner side for a third element; and wherein second protective element presents features which are similar to the first protective element with the variant that instead of having a hook shape it has a circular shape, ¼ of the circumference ending in a cupped coupling area to receive the proximal end of the first protective element.

43. The minimally invasive osteotomy device according to claim 40, wherein the third element or gripping element is shaped like a solid cube with a central hollow cavity in the form of a vertical slot which enables coupling of the cutting guide element.

44. The minimally invasive osteotomy device according to claim 40, wherein the cutting guide element comprises a solid circular plate with a full slot in its flange which can be adjusted, said slot has, in one section, a semicircular broadening at each side of its edges optionally working as a bit guide; and wherein the cutting guide element has two axes at the center of the outer sides of the plate, wherein the axes are coupled to the third element.

45. The minimally invasive osteotomy device according to claim 39, wherein the cutting element is selected from an oscillating sheet saw, bit and chisel.

46. The minimally invasive osteotomy device according to claim 30, comprising a single body having a grip handle extending from its distal end to approximately two thirds of the total length of said body; and a protective element extending from, wherein the grip handle ends at the proximal end of said body; a sliding element running across the interior of the handle; and a cutting guide element which is inserted into the inner side of the handle.

47. The minimally invasive osteotomy device according to claim 46, wherein the protective element is constituted by two sheets, an inner one and an outer one that are semicircular from a profile view and semi-tubular from a cross-sectional view, wherein the outer sheet has a concave oriented hitch area or flange, for the inner sheet to be able to slide without deviating from its lane; and wherein the inner sheet has a coupling fixed to the sliding element at its distal end and finishes in a blunt point in the form of an elevator at its proximal end.

48. The minimally invasive osteotomy device according to claim 46, wherein the sliding element is selected from a sling and strip, which runs across the interior of the handle, and having a thread or L-shaped quick coupling area, wherein when it is pushed inwards, it activates telescopic movement of the first sheet over the second one.

49. The minimally invasive osteotomy device according to claim 48, wherein the sliding element has at its distal end a mushroom-shaped rivet, which is housed in a cylindrical cavity where the most proximal side has a central opening through which the sling or strip passes, and the cap-shaped most distal side is followed by a handle coupling section, then a head with a central grip handle.

50. The minimally invasive osteotomy device according to claim 49, where the handle coupling section may be a threaded section when the coupling to the handle is threaded or a cylinder with a vertical arm when it is an L-shaped, quick coupling.

51. The minimally invasive osteotomy device according to claim 46, wherein the handle has a hollow, ergonomic, curved shape in its interior enabling the sliding element to run across its entire length, having at its distal end a threaded opening or an opening with an inner spring and an L-shaped slot in its wall, depending on whether the coupling of the sliding element is threaded or an L-shaped quick coupling.

52. The minimally invasive osteotomy device according to claim 51, wherein the grip handle has a hitch in its inner side for the cutting guide element.

53. The minimally invasive osteotomy device according to claim 46, wherein the cutting device is characterized by a solid cube with an inner adjustable slot having a fixed, vertical wall and another mobile wall which moves closer to or farther from the counter-side, thus increasing or reducing the vertical slot; and wherein the mobile wall has a mobile, bit guiding opening in the rotational plane.

54. The minimally invasive osteotomy device according to claim 30, formed by two symmetrical, Hohmann-type main bodies, constituted by a handle which is curved and concave on its outer side, having an area of coupling to the third element on its inner side; the handles of both main bodies are proximally followed by the protective elements; the protective elements have a curved shape from a side view, with inner concavity, and a semi-tubular shape from a cross-sectional view; wherein once both Hohmann-type main bodies are positioned around the bone, they are coupled to a third element which is the oscillating part of the sheet saw, which can make side rotational and cut deepening moves, generating a safe cut without the risk of going deeper than necessary or deviating.

55. The minimally invasive osteotomy device according to claim 30, wherein the device includes a spatial positioning element which makes it possible to orient, measure and regulate the bone structure cut in three spatial planes: frontal, sagittal and rotational; wherein the spatial positioning element is firmly secured to the bone structure to be cut, through at least one pair of outer fixation elements and is secured to the minimally invasive osteotomy device employing an anchoring mechanism.

56. The minimally invasive osteotomy device according to claim 55, wherein the spatial positioning element is formed by a straight arm and a curved arm; the horizontal, straight arm follows the bone axis, which is formed by two sheets and presents a central sliding channel running through its entire length, one of these sheets having an anchoring area for a Kirschner nail at one of its ends; the other sheet has, at its opposite end, a curved arm having a longitudinal central channel presenting an anchoring area for a Kirschner nail at one of its ends; and an anchoring device employed to secure the two sheets superimposed one another and the element for the spatial positioning to the osteotomy device; and wherein the ends of this device having anchoring sites that make it possible to use one, two or several Kirschner nails.

57. The minimally invasive osteotomy device according to claim 30, wherein the protection device may be incorporated to the cutting element, wherein the cutting element may be an oscillating sheet saw or a swing sheet saw.

58. The minimally invasive osteotomy device according to claim 30, wherein the tong-type protection device may be incorporated to the cutting element, in which the tong generates a side stop for the bone cut and an adjustable stop anterior to the bone plane preventing the deepening of the sheet saw.
